(19) United States
(12) Patent Application Publication
(10) Pub. No.: US 2006/0258870 A1
(43) Pub. Date: Nov. 16, 2006

(54) Process for producing bipyridinium compound

(76) Inventors: Masatomi Sen, Kanagawa (JP); Shingo Sato, Kanagawa (JP); Hideto Mori, Kanagawa (JP); Kazumi Nii, Kanagawa (JP)

Correspondence Address: SUGHRUE MION, PLLC, 2100 PENNSYLVANIA AVENUE, N.W., SUITE 800, WASHINGTON, DC, 20037, US

(21) Appl. No.: 10/556,672

(22) PCT Filed: Mar. 30, 2005
(86) PCT No.: PCT/JP05/06717

§ 371 Date: Nov. 14, 2005

(30) Foreign Application Priority Data
Apr. 2, 2004 (JP) 2004-110088
Mar. 24, 2005 (JP) 2005-086897

(51) Int. Cl. C07D 401/04

(52) U.S. Cl. 546/257

(57) ABSTRACT

A process for producing a bipyridinium compound represented by formula (4), the process comprises: reacting a bipyridine which may have a substituent with a halogenated (hetero)aryl compound represented by formula (1), so as to produce N,N′-bis((hetero)asyl)-bipyridimium compound represented by formula (2); and reacting the N,N′-bis((hetero)aryl)-bipyridinium compound with an amine compound represented by formula (3), without subjecting the N,N′-bis((hetero)aryl)-bipyridinium compound to an isolation treatment, In formula, R represents a (hetero)aryl group; X represents a halogen atom; R¹¹, R¹², R¹³, R¹⁴, R¹⁵, R¹⁶, R¹⁷ and R¹⁸ each independently represents a hydrogen atom or a substituent; and R¹ represents a (hetero)aryl group which may have a substituent or an alkyl group, wherein a polyhychic alcohol is used as a reaction solvent.

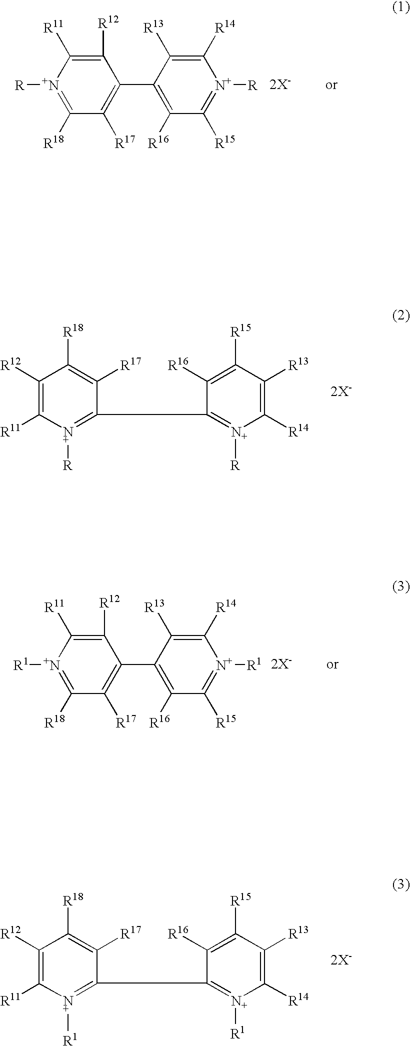

### TECHNICAL FIELD

The present invention relates to a process for producing a bipyridinium compound, preferably a 2,2′-bipyridinium compound and a 4,4′-bipyridinium compound useful as a herbicide or an electrochromic display material.

### Background Art

4,4′-bipyridinium compounds have already been put into practice as herbicides and, in recent years, they have been studied as electrochromic display materials. As a process for producing the 4,4′-bipyridinium compound, there has been a process called Menshutkin reaction. However, this reaction process fails to produce an aryl-substituted 4,4′-bipyridinium compound. As a process for producing the aryl-substituted 4,4′-bipyridinium compound, there is the following process (Bull. Chem. Soc. Jpn., 1991, vol.64, pp.321-323).

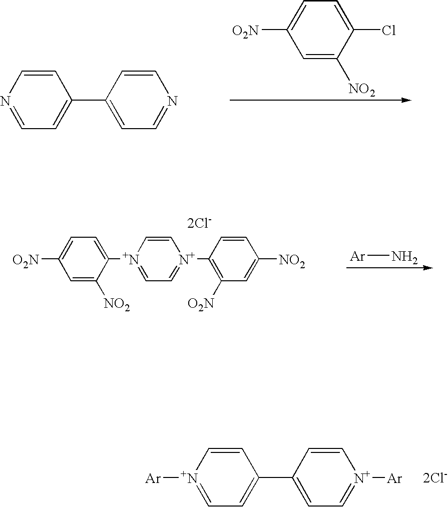

As another process for producing an aryl-substituted 4,4′-bipyridinium compound, there has been disclosed a process which involves a key step of reacting 4,4′-bipyridine with a halogenated hetero-aryl compound (JP-A-2003-128654).

On the other hand, a load by a process for producing a chemical product against environment has become a problem in recent years, and a clean chemical reaction which employs a mild reaction condition, which imposes a less load on environment and work and which minimizes the use of a harmful solvent and a reactant has been required (for example, Kagaku Furontia (Chemical Frontier), (4), Green Chemistry, Kagaku Dojin, translated by GSC Network, Nov. 30, 2001).

### DISCLOSURE OF THE INVENTION

The process described in above-mentioned Bull. Chem. Soc. Jpn., 1991, vol.64, pp.321-323 is difficultly said to be satisfactory, because the first step reaction between 4,4′-bipyridine and the halogenated aryl compound requires heating as long as 72 hours in acetonitrile, which leads to an increase in production cost. Further, the product of N,N′-bis(aryl)-4,4′-bipyridinium compound often stimulates or irritates skin, and therefore a special care must be taken in handling it. In addition, a study of the inventors has revealed that the process disclosed in JP-A-2003-128654 also requires a long period of time for the reaction between 4,4′-bipyridine and the halogenated hetero-aryl compound and that control of the reaction is difficult. In case when progress of the reaction is insufficient, N-hetero-arylation will be stopped at the stage where the N-hetero-arylation occurs at one position, which can be the cause of a decrease in the yield of the end product. On the other hand, an increase in the reaction temperature for the purpose of improving reaction ratio has resulted in progress of decomposition of the end product.

As is described above, the related processes for producing the 4,4′-bipyridinium compounds can never be said to be advantageous in consideration of yield, safety of the starting material or the intermediate, required reaction time and consideration on environment, and a technology which enables one to safely produce a highly pure 4,4′-bipyridinium compound under mild conditions and through simple procedures has strongly been demanded.

It is, therefore, an object of the invention to provide a process for producing a 4,4′-bipyridinium compound which can be conducted safely, efficiently and inexpensively on an industrial scale.

With the above circumstances in mind, the inventors have investigated the process for producing a bipyridinium compound, preferably a 4,4′-bipyridinium compound and, as a result, have unexpectedly found that the reaction can be made mild by employing a special reaction condition and that the end product can be produced continuously without isolating an intermediate, thus having achieved the present invention. That is, the object of the invention can be attained by the following means.

(1) A process for producing a bipyridinium compound represented by formula (4), the process comprises:

reacting a bipyridine which may have a substituent with a halogenated (hetero)aryl compound represented by formula (1), so as to produce a N,N′-bis((hetero)aryl)-bipyridinium compound represented by formula (2); and

reacting the N,N′-bis((hetero)aryl)-bipyridinium compound with an amine compound represented by formula (3), without subjecting the N,N′-bis((hetero)aryl)-bipyridinium compound to an isolation treatment,

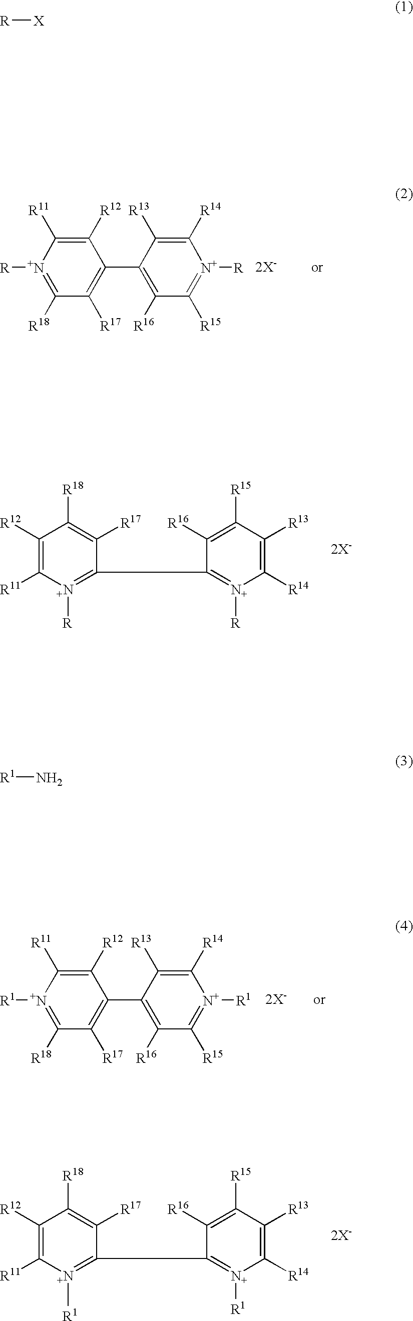

wherein R represents a (hetero)aryl group;

X represents a halogen atom;

R¹¹, R¹², R¹³, R¹⁴, R¹⁵, R¹⁶, R¹⁷ and R¹⁸ each independently represents a hydrogen atom or a substituent; and

R¹ represents a (hetero)aryl group which may have a substituent or an alkyl group,

wherein a polyhydric alcohol is used as a reaction solvent.

(2) The process as described in (1) above,

wherein the polyhydric alcohol is at least one of an ethylene glycol, a propylene glycol, a butylenes glycol, a glycerin, a diethylene glycol and a triethylene glycol.

(3) The process as described in (1) or (2) above,

Wherein a reaction of the bipyridine with the halogenated (hetero)aryl compound is conducted in the reaction solvent comprising the polyhydric alcohol so as to produce the reacted solution comprising the polyhydric alcohol, and the reacted solution is subjected to a reaction with the amine compound.

(4) The process as described in any of (1) to (3) above,

wherein an amount of the halogenated (hetero)aryl compound is 2.0 to 5.0 mols per mol of the bipyridine.

(5) The process as described in any of (1) to (4) above,

wherein a reaction of the bipyridine with the halogenated (hetero)aryl compound is conducted in a temperature of 60 to 150° C.

(6) The process as described in any of (1) to (5) above,

wherein a reaction of the bipyridine with the halogenated (hetero)aryl compound is conducted within 9 hours.

(7) A process for producing a bipyridinium compound represented by formula (4), the process comprises:

reacting a bipyridine which may have a substituent with a halogenated (hetero)aryl compound represented by formula (1) in a reaction solvent comprising a polyhydric alcohol, so as to produce a reacted solution comprising a N,N′-bis((hetero)aryl)-bipyridinium compound represented by formula (2); and

reacting the reacted solution comprising the N,N′-bis((hetero)aryl)-bipyridinium compound with an amine compound represented by formula (3),

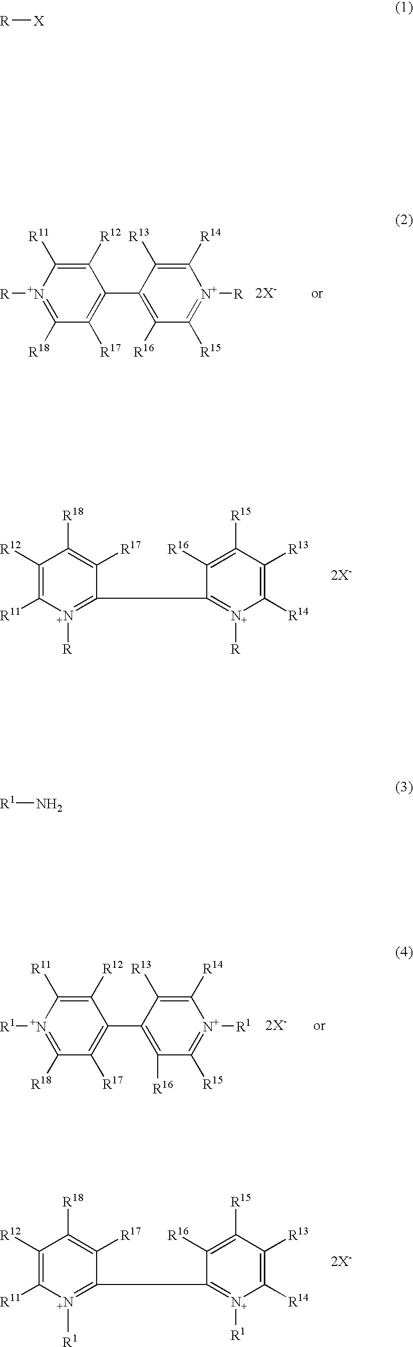

wherein R represents a (hetero)aryl group;

X represents a halogen atom;

R¹¹, R¹², R¹³, R¹⁴, R¹⁵, R¹⁶, R¹⁷ and R¹⁸ each independently represents a hydrogen atom or a substituent; and

R¹ represents a (hetero)aryl group which may have a substituent or an alkyl group.

### BEST MODE FOR CARRYING OUT THE INVENTION

First, the halogenated (hetero)aryl compound represented by formula (1) to be used in the process of the invention is described below. In formula (1), R represents a (hetero)aryl group. The term “(hetero)aryl group” as used herein means a cyclic residue having aromaticity, and may be either of an aryl group constituted by only carbon atoms and a hetero-aryl group containing a hetero atom such as N, O, S or Se. The aryl group and the hetero-aryl group include those which have a substituent or substituents.
R—X Formulat (1):

In the case where R is constituted by carbon atoms alone, the aryl group may have a substituent or substituents. Examples of the aryl group include a phenyl group and a naphthyl group. Of these, a phenyl group is more preferred, and a phenyl group having an electron attractive group on the ring is more preferred. Examples of the electron attractive group include a cyano group, a nitro group, an acyl group containing from 1 to 6 carbon atoms, an alkoxycarbonyl group containing from 1 to 6 carbon atoms and an alkylsulfonyl group containing from 1 to 6 carbon atoms. Among them, a cyano group, a nitro group and an alkylsulfonyl group containing from 1 to 6 carbon atoms are preferred. The most preferred examples of the substituent is a nitro group and, as R, a 2,4-dinitrophenyl group is most preferred.

In the case where R represents a hetero-aryl group containing a hetero atom such as N, O, S or Se, the hetero-aryl group contains preferably from 1 to 20 carbon atoms, more preferably from 3 to 10 carbon atoms, and examples thereof include an oxazole ring, a benzoxazole ring, a thiazole ring, a benzothiazole ring, an imidazole ring, a benzimidazole ring, a pyridine ring and a pyrimidine ring. Of these, a benzoxazole ring, a thiazole ring, a benzothiazole ring, an imidazole ring, a benzimidazole ring and a pyrimidine ring are preferred, with a thiazole ring, a benzothiazole ring and a pyrimidine ring being more preferred.

The above-described hetero-aryl group may have a substituent or substituents. In the case of using, in this specification, the phrase “may have a substituent or substituents” used with respect to a certain functional group (e.g., an aryl group, an aryloxy group or an alkyl group), the number and the kind of the substituent are not particularly limited and, when a plurality of substituents exist, they may be the same or different from each other. Examples of the substituent which can exist on the hetero-aryl group include a halogen atom, an alkyl group, a halo-alkyl group, an alkoxy group, a halo-alkoxy group, an aryl group, an aryloxy group, an alkenyl group, an alkynyl group, a cyano group, a hydroxyl group, a carboxyl group, a sulfo group, a dialkylamino group, an alkylthio group, an arylthio group, an alkylsulfonyl group, an arylsulfonyl group, a nitro group, a formyl group, an alkylcarbonyl group, an arylcarbonyl group, an alkoxycarfbonyl group, a carbamoyl group, a sulfamoyl group and an alkylenedioxy group. However, these are not limitatrive at all. Also, these substituents may further have one or more substituents. In such case, the above-illustrated substituents may favorably be used as the substituents. For example, a substituent containing an aryl group such as an arylalkyl group or an arylcarbonyl group may have from about 1 to about 5, preferably from 1 to 2, substituents such as an alkyl group, a halogen atom or an alkoxy group on the aryl ring.

In formula (1), X represents a halogen atom. The term “halogen atom” as used herein means any of a fluorine atom, a chlorine atom, a bromine atom and an iodine atom. In view of availability and price of the starting material, X represents preferably a chlorine atom or a bromine atom, more preferably a chlorine atom.

The N,N′-bis((hetero)aryl)-bipyridinium compound to be produced in the invention as an intermediate is represented by the following formula (2):

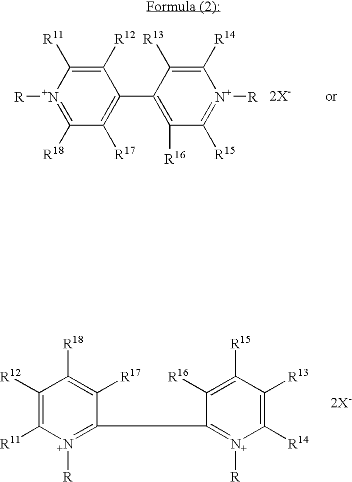

In formula (2), R¹¹, R¹², R¹³, R¹⁴, R¹⁵, R¹⁶, R¹⁷, R¹⁸ each independently represents a hydrogen atom or a substituent. Examples of the substituent include a halogen atom, an alkyl group, a halo-alkyl group, an alkoxy group, a halo-alkoxy group, an aryl group, an aryloxy group, an arylalkyl group, an alkenyl group, an alkynyl group, a cyano group, a hydroxyl group, a carboxyl group, a sulfo group, a dialkylamino group, an alkylthio group, an arylthio group, an alkylsulfonyl group, an arylsulfonyl group, a nitro group, a formyl group, an alkylcarbonyl group, an arylcarbonyl group, an alkoxycarbonyl group, a carbamoyl group, a sulfamoyl group and an alkylenedioxy group. However, these exmples do not limit the invention in any way. In the invention, those wherein all of R¹¹, R¹², R¹³, R¹⁴, R¹⁵, R¹⁶, R¹⁷ and R¹⁸ represent a hydrogen atom are more preferred. R and X are the same as defined with respect to formula (1), and preferred scopes thereof are also the same as described there.

Next, the amine compound represented by formula (3) are described below.
R¹—NH₂ Formula (3):

In formula (3), R1 represents a (hetero)aryl group or an alkyl group which may have a substituent. Examples of the (hetero)aryl group include the same as have been illustrated with respect to formula (1). Preferred examples thereof include a phenyl group, a 1-naphthyl group and a 2-naphthyl group. These may have a substituent, and examples of the substituent to be present include a halogen atom, an alkyl group, a halo-alkyl group (a halogen substituted alkyl group), an alkoxy group, a halo-alkoxy group (a halogen substituted alkoxy group), an aryl group, an aryloxy group, an arylalkyl group, an alkenyl group, an alkynyl group, a cyano group, a hydroxyl group, a carboxyl group, a sulfo group, a dialkylamino group, an alkylthio group, an arylthio group, an alkylsulfonyl group, an arylsulfonyl group, a nitro group, a formyl group, an alkylcarbonyl group, an arylcarbonyl group, an alkoxycarbonyl group, a carbamoyl group, a sulfamoyl group and an alkylenedioxy group. Of these, an alkyl group, an aryl group, an aryloxy group, an arylalkyl group, a hydroxyl group, a carboxyl group, a sulfo group, an alkylsulfonyl group, an arylsulfonyl group, a nitro group, an alkylcarbonyl group, an arylcarbonyl group, an alkoxycarbonyl group and a carbamoyl group are preferred, with an alkyl group, an aryl group, a hydroxyl group, a carboxyl group and a sulfo group being more preferred.

Examples of the alkyl group include alkyl groups containing from 1 to 22, preferably from 1 to 12, more preferably from 1 to 8, carbon atoms and having a straight-chain, branched or cyclic structure or the structure of the combination thereof. Preferred examples thereof include a straight-chain alkyl group, a branched alkyl group and a cyclic alkyl group. More specific examples thereof include an isopropyl group, a t-butyl group, a cyclohexyl group, a benzyl group and an adamantly group.

The bipyridinium compound to be produced by the production process of the invention is represented by the following formula (4).

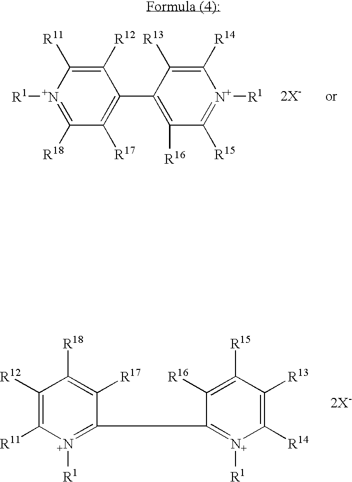

In formula (4), R¹¹, R¹², R¹³, R¹⁴, R¹⁵, R¹⁶, R¹⁷, R¹⁸, R¹ and X are the same as described with respect to formula (2), and preferred scopes thereof are also the same as described there. R¹ and X are the same as described with respect to formulae (1) and (3), and preferred scopes thereof are also the same as described there.

As preferred embodiments of the invention, specific examples of the compounds represented by formulae (1), (2), (3) and (4) are shown below which, however, are not to be construed as limiting the invention in any way. Specific examples of the compound represented by formula (1): 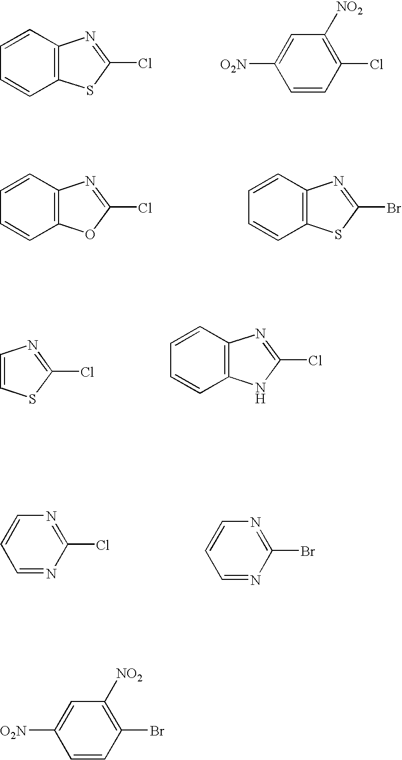
Specific examples of the compound represented by formula (2): 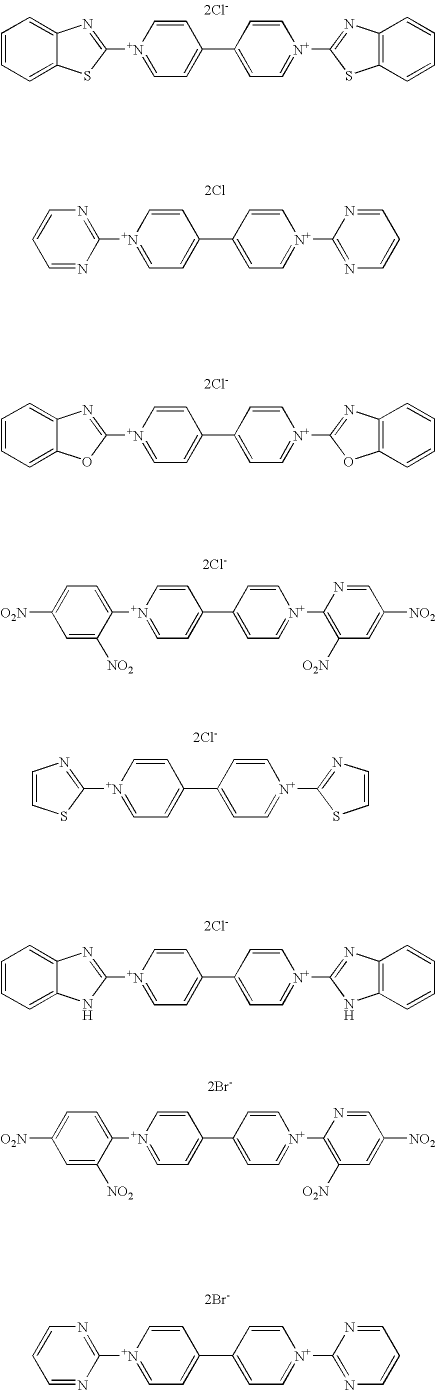
Specific examples of the compound represented by formula (3): 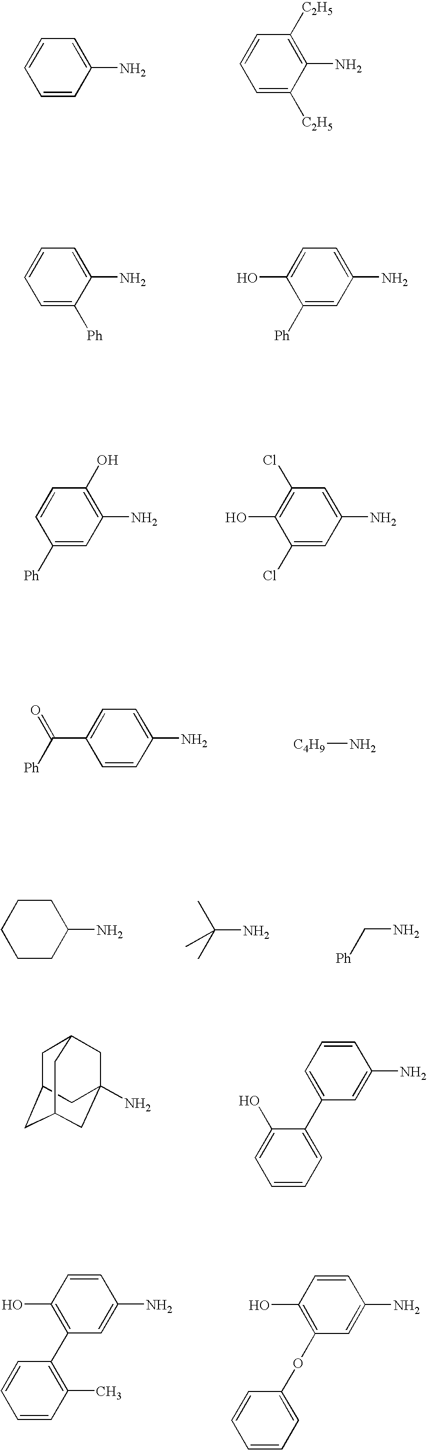 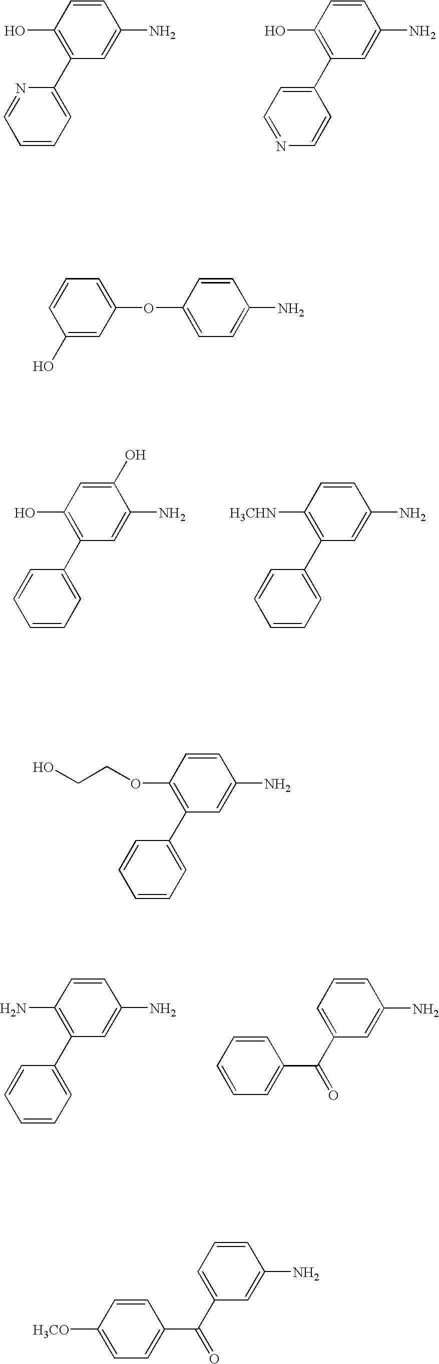 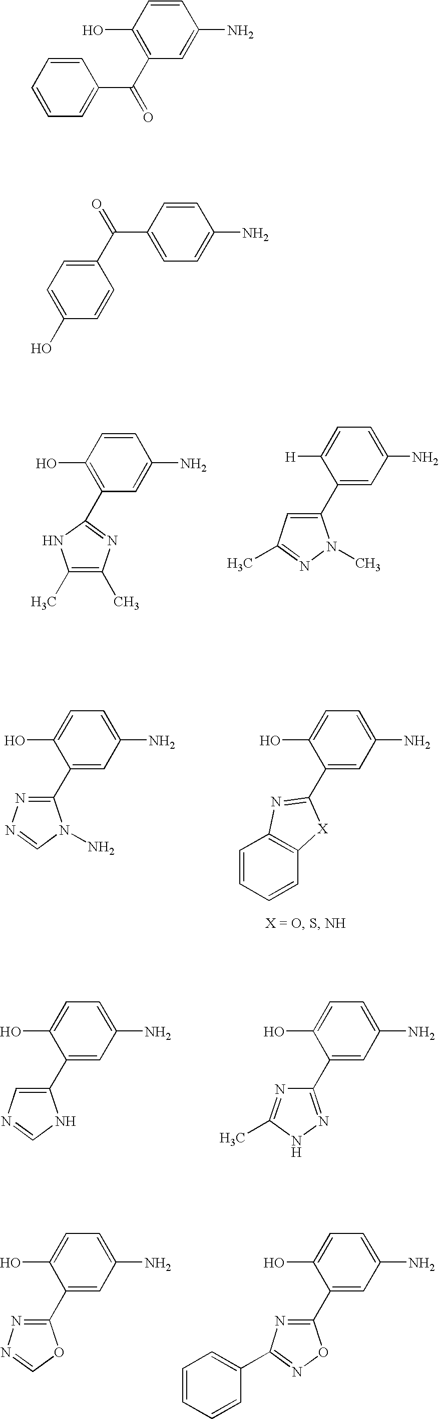 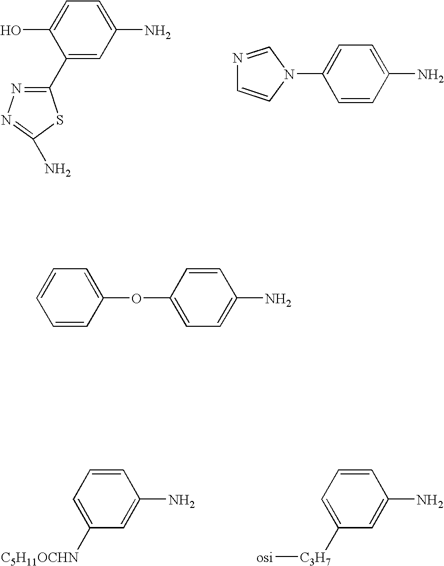
Specific examples of the compound represented by formula (4):

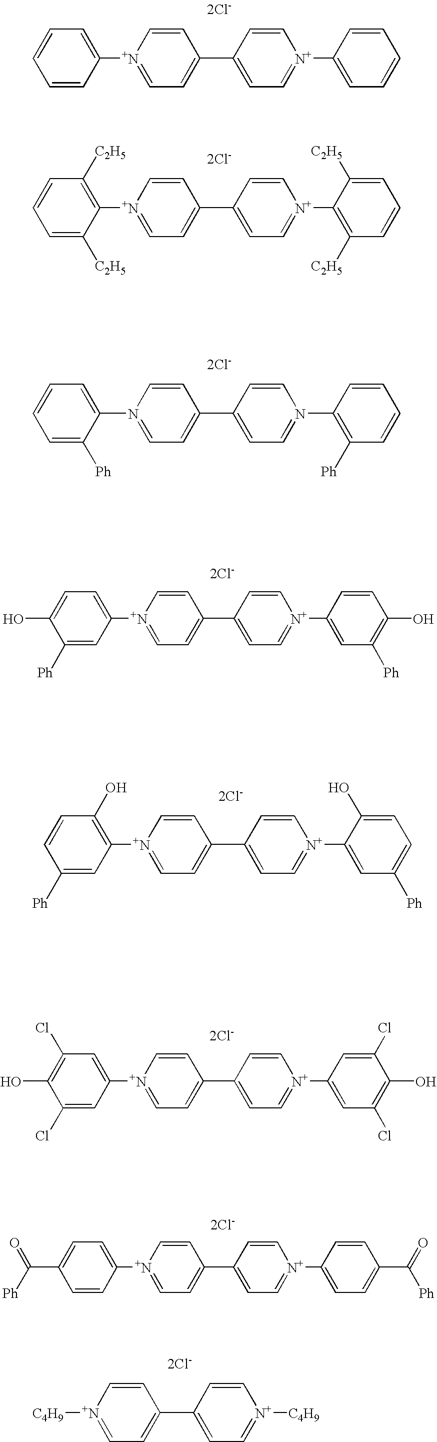

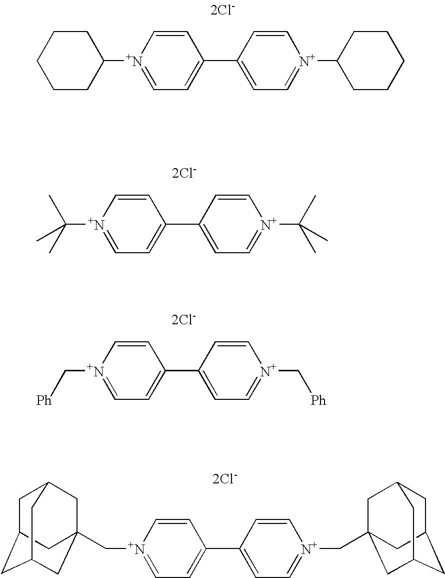

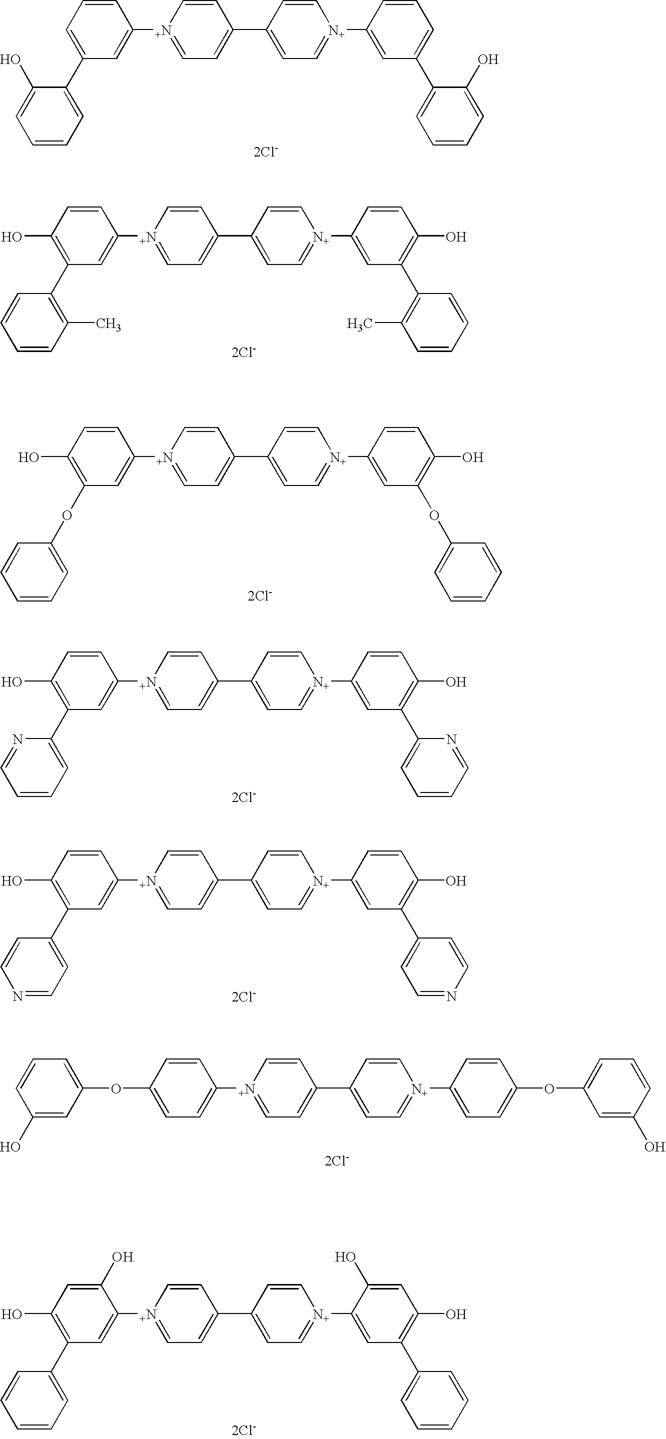

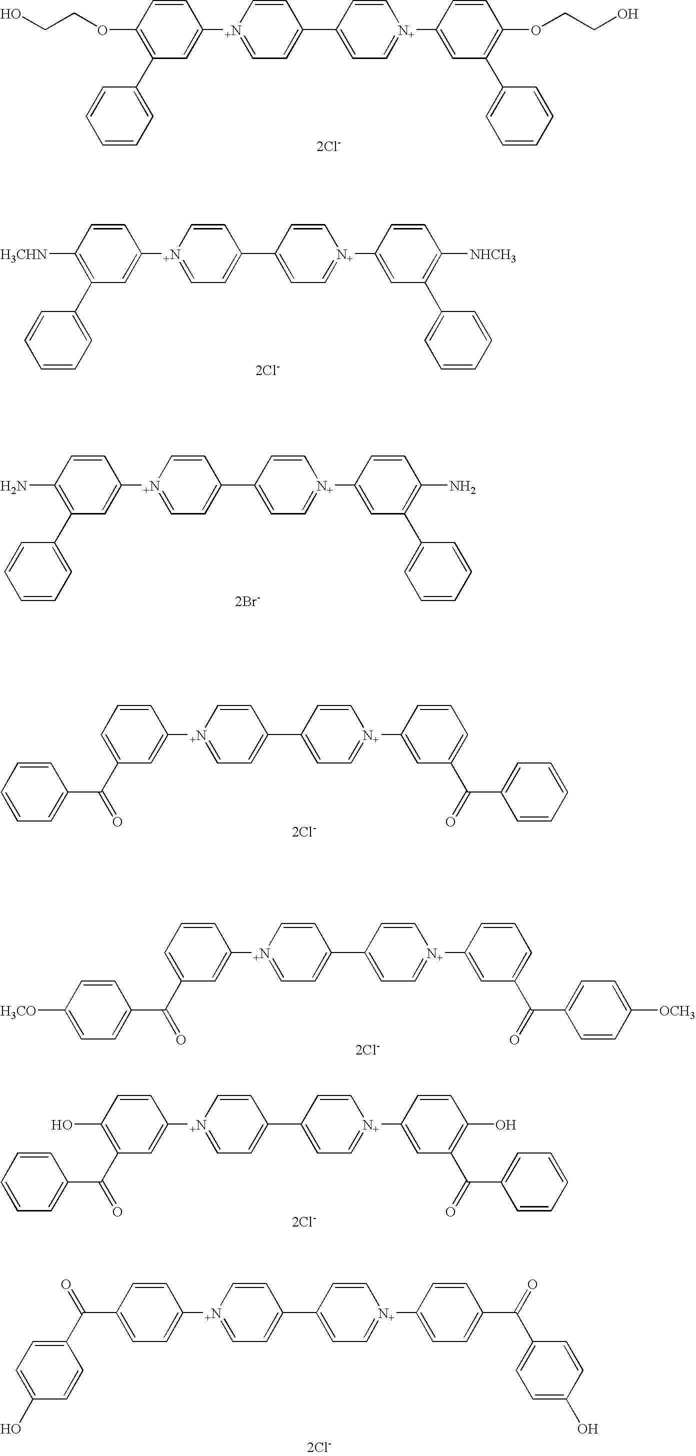

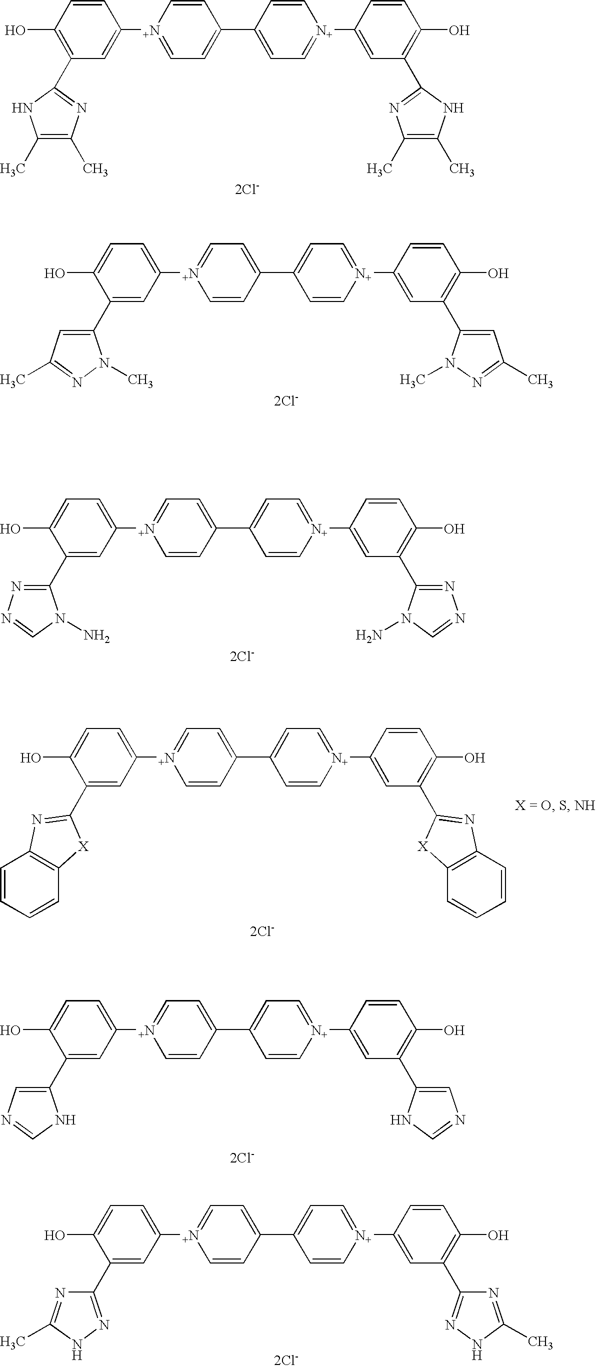

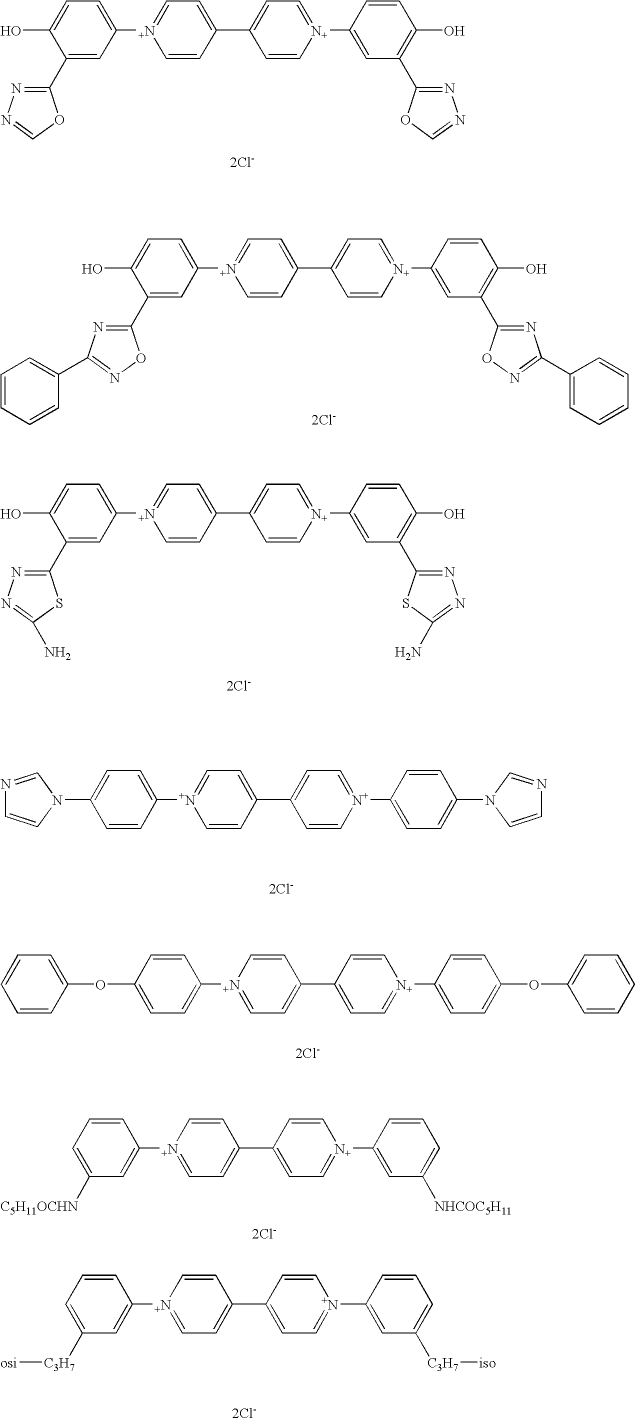

The production process of the invention is described in more detail below. The preferable production process of the invention comprises the first step of reacting 4,4′-bipyridine with the halogenated (hetero)aryl compound represented by formula (1) to produce the N,N′-(hetero)aryl-4,4′-bipyridinium compound represented by formula (2) and the second step of reacting the resultant N,N′-bis((hetero)aryl)-4,4′-bipyridinium compound with the amine compound represented by formula (3) to produce the 4,4′-bipyridinium compound represented by formula (4), and is characterized in that these two steps are conducted continuously with using a polyhydric alcohol as a reaction solvent. Preferred examples of the polyhydric alcohol include ethylene glycol, propylene glycol, butylenes glycol, glycerin, diethylene glycol and triethylene glycol. Of these, ethylene glycol, propylene glycol, glycerin, diethylene glycol and triethylene glycol are more preferred. The most preferred solvent is ethylene glycol, propylene glycol, diethylene glycol or a mixture of a plurality of solvents selected from these solvents.

In the first step of reacting the halogenated (hetero)aryl compound with 4,4′-bipyridine, the amount of the halogenated (hetero)aryl compound is in the range of from 2.0 to 10.0 mols per mol of 4,4′-bipyridine. The production ratio and production rate of the end product are not influenced so much by use of the large excess of the halogenated (hetero)aryl compound. However, use of the halogenated (hetero)aryl compound in a too excess amount imposes complicated after-treatment procedures, and leads to an increase in an amount of waste and an increase in production cost, thus being unfavorable in the production of the end product on an industrial scale. A preferred amount of the halogenated (hetero)aryl compound to be used in the invention is 2.0 to 5.0 mols per mol of 4,4′-bipyridine, a more preferred amount thereof is 2.0 to 3.5 mols, and a still more preferred amount thereof is 2.0 to 3.2 mols.

The reaction between the halogenated (hetero)aryl compound and 4,4′-bipyridine is conducted at a temperature of from 10 to 180° C., preferably from 60 to 150° C., still more preferably from 80 to 140° C. The reaction time varies depending upon the amounts of charged reactants and the reaction temperature, but is usually within 9 hours which is much shorter than the reaction time required for the process described in above-mentioned Bull. Chem. Soc. Jpn., 1991, vol.64, pp.321-323. Upon conducting the reaction, an inert atmosphere is not particularly necessary, but the reaction may be conducted in a stream of argon or nitrogen.

The reason why the polyhydric alcohols of the invention are effective is not clear. However, it can be considered as followings. First, a dihydric alcohol or a trihydric alcohol has a higher boiling point than that of a monohydric alcohol having the same number of carbons atoms as the. dihydric alcohol or the trihydric alcohol, therefore the temperature of the reaction solution comprising the dihydric alcohol or the trihydric alcohol can be much increased. Second, the dihydric alcohol or the trihydric alcohol is easier to solve the bipyridinium compound than the monohydric alcohol due to a plurality of polar OH groups that coordinates the divalent cation part of the bipyridinium compound. From these considerations, the effect of the activation for the reaction or the solubility of the bipyridinium compound etc. is presumed.

The N,N′-bis((hetero)aryl-4,4′-bipyridinium compound obtained by the above-described step can be continuously used, without isolation, in the subsequent second step, which is an industrially advantageous point in view of efficiency, production cost and safety.

In the step of reacting the N,N′-bis((hetero)aryl)-4,4′-bipyridinium compound with the amine compound represented by formula (3) to produce the 4,4′-bipiridinium compound represented by formula (4), the amount of the amine compound to be used is in the range of from 2.0 to 10.0 mols per mol of the N,N′-bis((hetero)aryl-4,4′-bipyridinium compound but, as is the same with the first step, use of a large excess of the amine compound does not influence the production ratio/production rate improvement so much. Use of the amine compound in a too excess amount leads to an increase in an amount of waste and an increase in production cost, thus being unfavorable in the production of the end product on an industrial scale. A preferred amount of the amine compound to be used in the invention is 2.0 to 5.0 mols per mol of the N,N′-bis((hetero)aryl-4,4′-bipyridiinium compound, a more preferred amount thereof is 2.0 to 3.5 mols, and a still more preferred amount thereof is 2.0 to 3.0 mols. As the reaction solvent, the solvent having been used in the first step can be used in common. The scope of preferably usable polyhydric alcohol is the same as that having been described hereinbefore. Also, it is possible to use an auxiliary solvent in view of industrial procedure such as improvement of fluidity of the reaction mixture or improvement of stirring efficiency. Preferred examples of the auxiliary solvent include methanol, ethanol, 2-propyl alcohol, acetone, methyl ethyl ketone, N,N-dimethylformamide and N,N-dimethylacetamide. These may be used in combination thereof.

The reaction between the N,N′-bis((hetero)aryl)-4,4′-bipyridinium compound and the amine compound is conducted at a temperature of from 10 to 180° C., preferably from 20 to 120° C., still more preferably from 20 to 90° C. The reaction time varies depending upon the amounts of charged reactants and the reaction temperature, but is usually within 6 hours. Upon conducting the reaction, an inert atmosphere is not particularly necessary, but the reaction may be conducted in a stream of argon or nitrogen.

As a method for isolating an end product from the reaction mixture after completion of the invention, it is possible to apply an ordinary separating and purifying means. For example, it is possible to employ a method of adding a poor solvent to the reaction mixture, cooling the mixture to precipitate the end product as crystals and isolating the end product by the ordinary solid-liquid separating technique. The 4,4′-bipyridinium compounds obtained as described above usually have an enough high purity to be used in the subsequent step without conducting further purification. However, for some use or purpose, further purification may be conducted. As such purification method, there may be applied those methods which are usually employed for purifying an organic compound, such as recrystallization or slurry suspension purification using an organic solvent such as methanol, ethanol, 2-propyl alcohol, acetone, methyl ethyl ketone, N,N-dimethylformamide or N,N-dimethylacetamide.

### EXAMPLES

The invention is described in more detail by reference to Examples and Comparative Examples which, however, are not to be construed as limiting the invention in any way.

### Example 1

### Synthesis of Compound 1

The synthesis scheme is shown below.

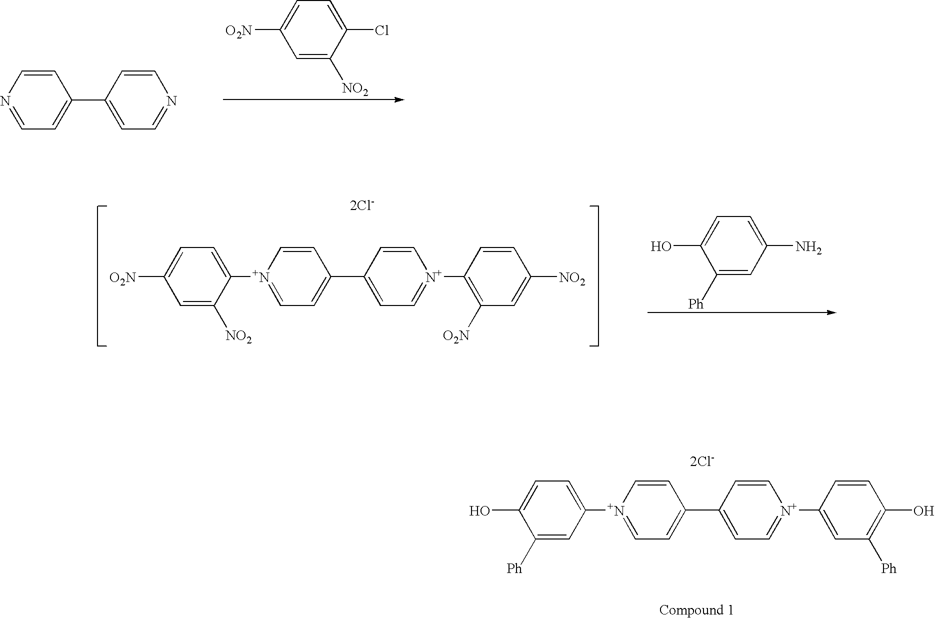

4,4′-Bipyridine (23.4 g, 0.15 mol) and 2,4-dinitrochlorobenzene (76.0 g, 0.375 mol) were dissolved in ethylene glycol (300 mL), and the reaction mixture was stirred for 6 hours at an inside temperature of 120° C. Analysis of the reaction mixture revealed that the ratio of the starting material: mono-substituted product: bis-substituted product (end product) was 1:29:70 (in terms of HPLC areal intensity ratio). After cooling the reaction mixture to 90° C., 4-hydroxy-3-phenylaniline (55.6 g, 0.30 mol) was added to the reaction mixture, followed by stirring the reaction mixture for 3 hours at an inside temperature of 80° C. Acetone (300 mL) was added thereto and, after refluxing for 30 minutes under heating, the reaction mixture was cooled to an inside temperature of 5° C. to precipitate crystals. After stirring the mixture for 2 hours at 5° C., the crystals were collected by filtration, washed with acetone and dried to obtain the end product of compound 1 as orange crystalline powder.

Yield: 56.0 g; 66%

¹H-NMR (TMS, CD3OD); δ 9.54 (d, 4H, pyridine ring moiety), 8.86 (d, 4H, pyridine ring moiety), 7.25-7.86 (m, 18H).

mp: 250° C. or above (decomposition)

### Comparative Example 1

Results of Conducting the Reaction for Synthesizing Compound 1 Using Acetonitrile:

The synthesizing scheme is the same as described in Example 1.

4,4′-Bipyridine (1.6 g) and 2,4-dinitrochlorobenzene (7.0 g; 3.5 mols per mol of 4,4′-bipyridine) were suspended in acetonitrile (35 mL), and the reaction mixture was refluxed for 48 hours under heating. Analysis of the reaction mixture by HPLC after 48-hour reaction revealed that the ratio of the starting material: mono-substituted product: bis-substituted product was 14:85:1 (in terms of HPLC areal intensity ratio). The reaction did not proceed any more when the reaction was further continued by refluxing under the condition of heating for a total period of 72 hours. Then, 4-hydroxy-3-phenylaniline (6.6 g; 3.5 mols per mol of 4,4′-bipyridine) was added to the reaction mixture, followed by refluxing under heating for 12 hours. However, formation of the end product of compound 1 was not observed.

Additionally, at the point of reacting 4,4′-bipyridine with 2,4-dinitrochlorobenzene, crystals separated out from the reaction mixture. As a result of separate analysis of the crystals, they were found to be crystals of N-mono-aryl derivative. That is, it is surmised that, under the above-described conditions, the intermediate separates out as crystals and are removed out of the system, thus the reaction to form the bis derivative being difficult to proceed.

### Comparative Example 2

Results of Conducting the Reaction for Synthesizing Compound 1 Using Dimethylformamide (DMF):

The synthesizing scheme is the same as described in Example 1.

4,4′-Bipyridine (1.6 g) and 2,4-dinitrochlorobenzene (7.0 g; 3.5 mols per mol of 4,4′-bipyridine) were suspended in DMF (30 mL), and the reaction mixture was stirred at 120° C. for 20 hours. Analysis of the reaction mixture by HPLC after 20-hour reaction revealed that the ratio of the starting material: mono-substituted product: bis-substituted product was 13:84:3 (in terms of HPLC areal intensity ratio). After further continuing the reaction for a total period of 36 hours, 4-hydroxy-3-phenylaniline (6.6 g; 3.5 mols per mol of 4,4′-bipyridine) was added to the reaction mixture, followed by conducting the reaction for 12 hours. However, formation of the end product of compound 1 was scarcely observed, thus further procedures being stopped.

### Comparative Example 3

Results of Conducting the Reaction for Synthesizing Compound 1 Using Methanol:

The synthesizing scheme is the same as described in Example 1.

4,4′-Bipyridine (1.6 g) and 2,4-dinitrochlorobenzene (7.0 g; 3.5 mols per mol of 4,4′-bipyridine) were suspended in methanol (35 mL), and the reaction mixture was refluxed for 20 hours under heating. Analysis of the reaction mixture by HPLC after 20-hour reaction revealed that the ratio of the starting material: mono-substituted product: bis-substituted product was 1:85:14 (in terms of HPLC areal intensity ratio). After further continuing the reaction for a total period of 36 hours under the same condition, 4-hydroxy-3-phenylaniline (6.6 g; 3.5 mols per mol of 4,4′-bipyridine) was added to the reaction mixture, followed by conducting the reaction for 12 hours. However, after-treatment of the reaction mixture failed to isolate crystals of the end product of compound 1.

### Example 2

### Synthesis of Compound 2

The synthesis scheme is shown below.

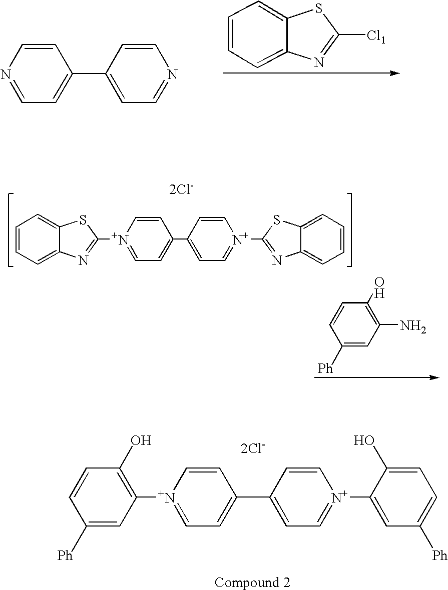

4,4′-Bipyridine (7.8 g, 0.05 mol) and 2-chlorobenzothiazole (29.6 g, 0.125 mol) were dissolved in ethylene glycol (120 mL), and the reaction mixture was stirred for 8 hours at an inside temperature of 120° C. To this reaction solution was added 2-hydroxy-5-phenylaniline (18.5 g, 0.10 mol), and the resulting reaction mixture was stirred for 5 hours at an inside temperature of 80° C. Acetone (150 mL) was added thereto and, after refluxing for 30 minutes under heating, the reaction mixture was cooled to an inside temperature of 15° C. to precipitate crystals. After stirring the mixture for 2 hours at 5° C., the crystals were collected by filtration, washed with acetone and dried to obtain the end product of compound 2 as orange crystalline powder.

Yield: 17.3 g; 61%

¹H-NMR (TMS, CD3OD); δ 9.83 (d, 4H, pyridine ring moiety), 8.42 (d, 4H, pyridine ring moiety), 7.12-7.98 (m, 18H).

mp: 250° C. or above (decomposition)

### Comparative Example 4

Results of Conducting the Reaction for Synthesizing Compound 2 Using Acetonitrile:

The synthesizing scheme is the same as described in Example 2.

4,4′-Bipyridine (1.6 g) and 2-chlorobenzothiazole (6.1 g; 3.5 mols per mol of 4,4′-bipyridine) were suspended in acetonitrile (35 mL), and the reaction mixture was refluxed for 40 hours under heating. Analysis of the reaction mixture by HPLC after 48-hour reaction revealed that the ratio of the starting material: mono-substituted product: bis-substituted product was 21:76:3 (in terms of HPLC areal intensity ratio). To this reaction mixture was added 4-hydroxy-3-phenylaniline (6.6 g; 3.5 mols per mol of 4,4′-bipyridine), followed by refluxing for 12 hours under heating. After-treatment of the reaction mixture failed to isolate crystals of the end product of compound 2.

### Example 3

### Synthesis of Compound 3

The synthesis scheme is shown below.

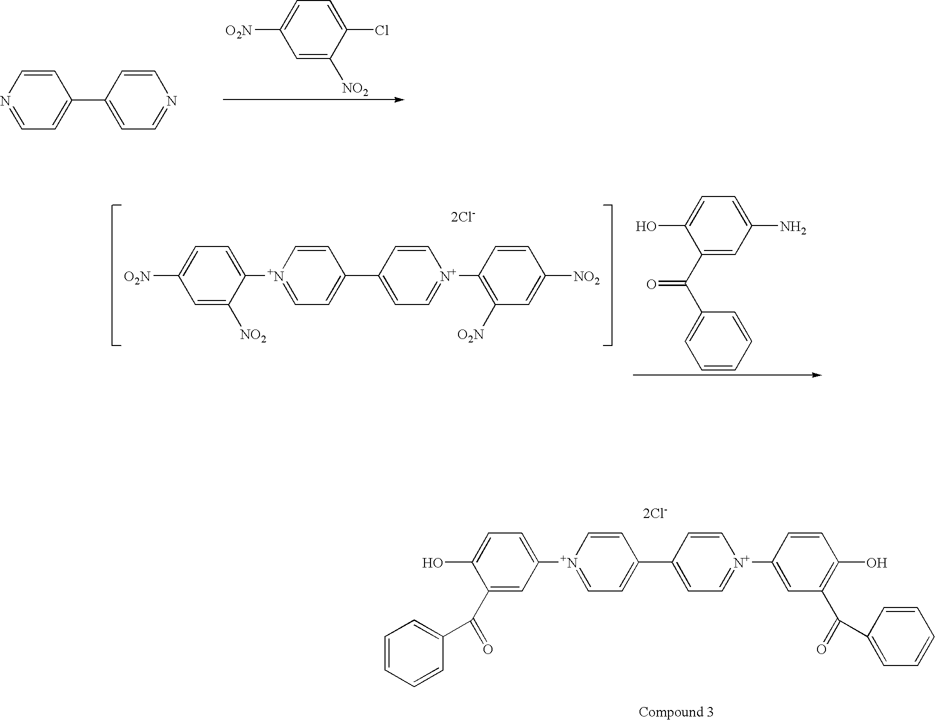

4,4′-Bipyridine (15.6 g, 0.10 mol) and 2,4-dinitrochlorobenzen (50.7 g, 0.25 mol) were dissolved in ethylene glycol (200 mL), and the reaction mixture was stirred for 6 hours at an inside temperature of 120° C. To this reaction solution was added 4-hydroxy-3-phenylcarbonylaniline (42.7 g, 0.20 mol), and the resulting reaction mixture was stirred for 6 hours at an inside temperature of 90° C. Acetone (250 mL) was added thereto and, after refluxing for 30 minutes under heating, the reaction mixture was cooled to an inside temperature of 5° C. and, after stirring the mixture for 1 hour at 10° C., the crystals were collected by filtration, washed with acetone and dried to obtain the end product of compound 3 as dark orange crystalline powder. Yield: 45.3 g; 72.9%

¹H-NMR (TMS, DMSO-d6); δ 9.65 (d, 4H, pyridine ring moiety), 9.04 (d, 4H, pyridine ring moiety), 7.38-8.01 (m, 16H), 11.31 (s, 2H, phenolic OH). mp: 250° C. or above (decomposition)

### Comparative Example 5

Results of Conducting the Reaction for Synthesizing Compound 3 Using Acetonitrile:

The synthesizing scheme is the same as described in Example 3.

4,4′-Bipyridine (3.12 g) and 2,4-dinitrochlorobenzene (10.14 g; 2.5 mols per mol of 4,4′-bipyridine) were suspended in acetonitrile (60 mL), and the reaction mixture was refluxed for 48 hours under heating. Analysis of the reaction mixture by HPLC after 48-hour reaction revealed that the ratio of the starting material: mono-substituted product: bis-substituted product was 12:84:2 (in terms of HPLC areal intensity ratio). The reaction did not proceed any more when the reaction was further continued by refluxing under the condition of heating for a total period of 72 hours. Then, 4-hydroxy-3-phenylcarbonylaniline (10.7 g; 2.5 mols per mol of 4,4′-bipyridine) was added to the reaction mixture, followed by refluxing under heating for 12 hours. However, formation of the end product of compound 3 was observed as a degree of about 1% (in terms of HPLC areal intensity ratio).

It can be seen from the results of Examples and Comparative Examples that the production process of the invention permits to shorten the reaction time and improve yield and enables one to obtain the end product continuously without isolating the intermediate, thus the production steps being simplified. Accordingly, superiority and usefulness of the production process of the invention is apparent.

### INDUSTRIAL APPLICABILITY

The process of the invention enables one to produce a bipyridinium compound, preferably a 4,4′-bipyridinium compound useful as a herbicide or an electrochromic display material safely, effectively and inexpensively on an industrial scale.

The entire disclosure of each and every foreign patent application from which the benefit of foreign priority has been claimed in the present application is incorporated herein by reference, as if fully set forth.

1. A process for producing a bipyridinium compound represented by formula (4), the process comprises:
reacting a bipyridine which may have a substituent with a halogenated (hetero)aryl compound represented by formula (1), so as to produce a N,N′-bis((hetero)aryl)-bipyridinium compound represented by formula (2); and
reacting the N,N′-bis((hetero)aryl)-bipyridinium compound with an amine compound represented by formula (3), without subjecting the N,N′-bis((hetero)aryl)-bipyridinium compound to an isolation treatment, 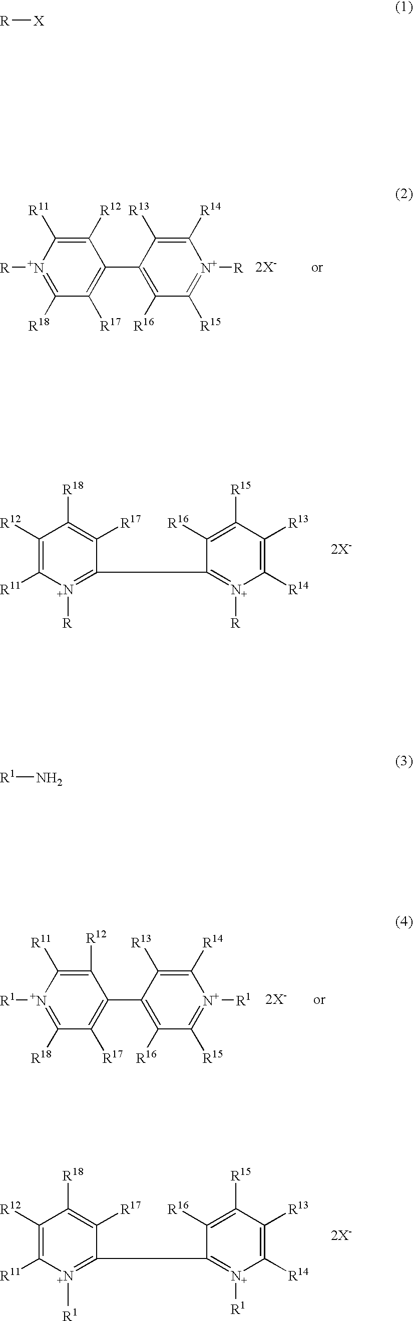
wherein R represents a (hetero)aryl group;
X represents a halogen atom;
R¹¹, R¹², R¹³, R¹⁴, R¹⁵, R¹⁶, R¹⁷ and R¹⁸ each independently represents a hydrogen atom or a substituent; and
R¹ represents a (hetero)aryl group which may have a substittent or an alkyl group,
wherein a polyhydric alcohol is used as a reaction solvent.

2. The process according to claim 1,
wherein the polyhydric alcohol is at least one of an ethylene glycol, a propylene glycol, a butylenes glycol, a glycerin, a diethylene glycol and a triethylene glycol.

3. The process according to claim 1,
Wherein a reaction of the bipyridine with the halpgenated (hetero)aryl compound is conducted in the reaction solvent comprising the polyhydric alcohol so as to produce the reacted solution comprising the polyhydric alcohol, and the reacted solution is subjected to a reaction with the amine compound.

4. The process according to claim 1,
wherein an amount of the halogenated (hetero)aryl compound is 2.0 to 5.0 mols per mol of the bipyridine.

5. The process according to claim 1,
wherein a reaction of the bipyridine with the halogenated (hetero)aryl compound is conducted in a temperature of 60 to 150° C.

6. The process according to claim 1,
wherein a reaction of the bipyridine with the halogenated (hetero)aryl compound is conducted within 9 hours.

7. A process for producing a bipyridinium compound represented by formula (4), the process comprises:
reacting a bipyridine which may have a substituent with a halogenated (hetero)aryl compound represented by formula (1) in a reaction solvent comprising a polyhydric alcohol, so as to produce a reacted solution comprising a N,N′-bis((hetero)aryl)-bipyridinium compound represented by formula (2); and
reacting the reacted solution comprising the N,N′-bis((hetero)aryl)-bipyridinium compound with an amine compound represented by formula (3), 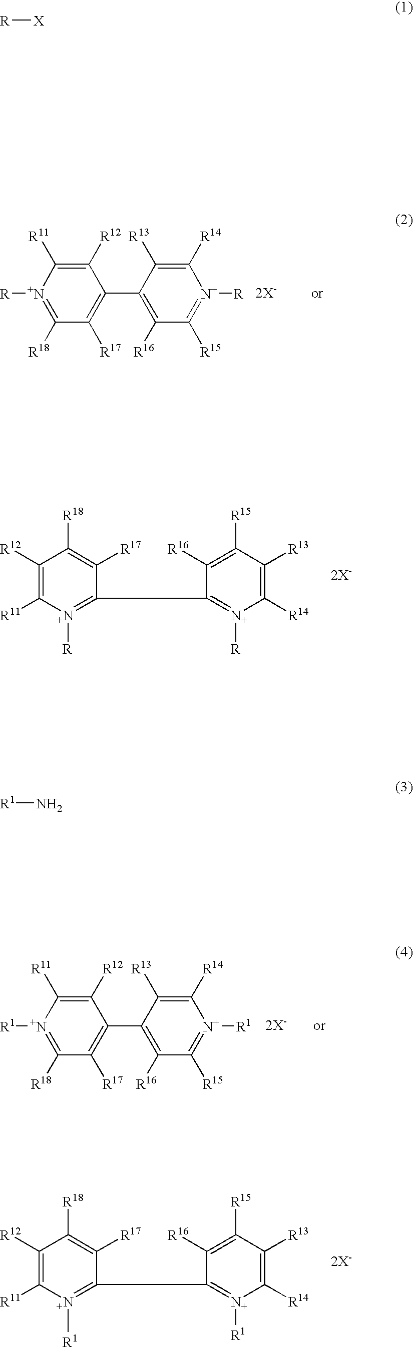
wherein R represents a (hetero)aryl group;
X represents a halogen atom;
R¹¹, R¹², R¹³, R¹⁴, R¹⁵, R¹⁶, R¹⁷ and R¹⁸ each independently represents a hydrogen atom or a substituent; and
R¹ represents a (hetero)aryl group which may have a substituent or an alkyl group.
